(19) United States
(12) Patent Application Publication
(10) Pub. No.: US 2021/0292270 A1
(43) Pub. Date: Sep. 23, 2021

(54) METHOD FOR OBTAINING PURE CYCLOHEXYL(METH)ACRYLATE BY DISTILLATION

(71) Applicant: BASF SE, Ludwigshafen am Rhein (DE)

(72) Inventors: Ortmund LANG, Ludwigshafen am Rhein (DE); Nadja Isabelle Desiree KLIPFEL, Sion (CH); Nicole JANSSEN, Ludwigshafen am Rhein (DE); Roland POX, Ludwigshafen am Rhein (DE)

(21) Appl. No.: 16/468,823

(22) PCT Filed: Dec. 13, 2017
(86) PCT No.: PCT/EP2017/082695

§ 371 Date: Jun. 12, 2019

(30) Foreign Application Priority Data
Dec. 13, 2016 (EP) 16203787.3

(51) Int. Cl. C07C 67/54; B01D 3/14; B01D 3/42

(52) U.S. Cl. CPC C07C 67/54; B01D 3/4266; B01D 3/14

(57) ABSTRACT

Process for isolating pure cyclohexyl (meth)acrylate from a mixture comprising cyclohexyl (meth)acrylate, cyclohexanol and entrainer, by feeding or introducing the mixture is fed as side feed stream into a rectification column having a bottom vaporizer, separation-active internals and a stripping section and an enrichment section, where the column has from 4 to 50 theoretical plates, the side feed stream is introduced in the region commencing at least one theoretical plate above the bottommost theoretical plate and ending at least one theoretical plate below the uppermost theoretical plate, the pressure at the top of the column is in the range from 10 mbar to 5 bar and the reflux ratio is from 1:0.2 to 1:10, and the bottom output is fed to a further distillation unit in which the cyclohexyl (meth)acrylate is separated off at the top or at a side offtake.

The present invention relates to a process for isolating pure cyclohexyl (meth)acrylate from a mixture comprising cyclohexyl (meth)acrylate, cyclohexanol and entrainer by distillation.

The term (meth)acrylic acid refers in a known manner to acrylic acid and/or methacrylic acid. Analogously, cyclohexyl (meth)acrylate means cyclohexyl acrylate and/or cyclohexyl methacrylate.

Cyclohexyl (meth)acrylate is a specialty monomer for the surface coatings and paint sector. Important applications are, for example, top clear coats for automobile coatings, resins for solvent-comprising and solvent-free surface coatings, weathering-resistant exterior emulsion paints and also adhesives.

Known processes for the industrial preparation of cyclohexyl (meth)acrylate are predominantly based on transesterification of a (meth)acrylate, in particular methyl (meth)acrylate, with cyclohexanol in the presence of a catalyst. A disadvantage of the transesterification processes for preparing cyclohexyl (meth)acrylate is that they are unfavorable for kinetic reasons, with correspondingly low space-time yields.

A process for the esterification of (meth)acrylic acid with cyclohexanol is described in EP 1 853 546 A1 (WO 2006/087297 A1), BASF AG.

In this process, after the esterification, the low boilers are firstly separated off by distillation (at the top) and the high boilers are subsequently separated off (at the bottom) from the crude product. In the low boiler distillation, the entrainer is firstly distilled off at the top (“process step D”). The alcohol is subsequently separated off at the top of a column and the target ester is separated off at the bottom (“process step E”). The steps “D” and “E” can also be carried out in a joint distillation unit.

The distillate stream, which generally comprises still relatively large amounts of target esters in addition to the alcohol separated off, is recirculated to the esterification stage (cf. scheme 1/1 in WO 06/087297).

The recirculation of product not only leads to a decrease in the total capacity of the plant but also has an adverse effect on the esterification conversion.

Particularly when a falling film evaporator is operated in the countercurrent mode as bottom vaporizer in “process step A”, quality problems in respect of the product of value (target ester) can occur because of the vaporizer type used in the plant. In addition, the total capacity of the plant is limited by the countercurrent mode of operation. Under the indicated operating conditions, backing-up of the downward-flowing liquid mixture can occur due to the upward-flowing vapor mixture. At a correspondingly, high held-up liquid column, sudden downflow of the liquid occurs as a result of the hydrostatic pressure and holding-up then occurs again. This leads to pulsation in the column. Stable operation of the distillation column can be greatly limited by the pulsation in this countercurrent mode of operation in the falling film evaporator.

Separation of the alcohol from the target ester may no longer be possible in the case of a further increase in capacity, the alcohol goes into the bottom region of the column and the product quality may no longer be ensured.

The process described thus has, in particular, the disadvantage that relatively large amounts of target ester are conveyed back into the esterification pot due to the recirculation of the distillate to the esterification. The thermal stress to which the product is again subjected increases the formation of secondary components. As a result, the product yield is reduced and the quality is decreased. The energy consumption is particularly high because of the multiple distillation of the target ester. A further disadvantage is the operation of the falling film evaporator in the countercurrent mode. Instabilities during operation of the column, fouling and an associated shorter time of operation of the vaporizer are the consequences.

Ullmann's Encyclopedia of Industrial Chemistry, Vol. A 1, chapter ‘Acrylic Acid and Derivatives’, Verlag VCH 1985, describes, on pages 161 to 176, the work-up by distillation of higher alkyl acrylates from an esterification reaction (FIG. 5, page 168). According to this book, the solvent is separated off at the top of a column f, the unreacted alcohol is subsequently distilled off in a side feed stream column g and the product ester is finally subjected to pure distillation in the column h.

It was an object of the present invention to provide an improved process for preparing cyclohexyl (meth)acrylate from (meth)acrylic acid and cyclohexanol, which, while adhering to the required specifications for the target product, does not have the disadvantages of the prior art and in particular provides a high-quality target ester in an energetically favorable process.

We have accordingly found a process for isolating pure cyclohexyl (meth)acrylate from a mixture comprising cyclohexyl (meth)acrylate, cyclohexanol and entrainer by distillation, wherein the mixture is fed as side feed stream into a rectification column having a bottom vaporizer, separation-active internals and a stripping section and an enrichment section, where the column has from 4 to 50 theoretical plates, the side feed stream is introduced in the region commencing at least one theoretical plate above the bottommost theoretical plate and ending at least one theoretical plate below the uppermost theoretical plate, the pressure at the top of the column is in the range from 10 mbar to 5 bar and the reflux ratio is from 1:0.2 to 1:10, and the bottom output is fed to a further distillation unit in which the cyclohexyl (meth)acrylate is separated off at the top or at a side offtake.

The process is preferably carried out continuously.

The entrainer (for water) is preferably cyclohexane.

In the process of the invention, the unreacted alcohol, i.e. the cyclohexanol, and further components having boiling points lower than the desired product are distilled off at the top in a specific step in a column having a side feed (side feed stream column). The desired product, i.e. the cyclohexyl (meth)acrylate, is fed via a bottom offtake to a subsequent distillation unit.

It has surprisingly been found that, as a result of the specific configuration and specific conditions of the side feed stream column in the removal of the low boilers, namely in particular cyclohexanol and entrainer, by distillation, the target ester, i.e. the cyclohexyl (meth)acrylate, can be very greatly depleted in the distillate.

In this way, the recycle stream into the first esterification stage is at the same time greatly reduced and, in addition, the capacity of a plant for preparing cyclohexyl (meth)acrylate is significantly increased and the conversion of the reaction is also increased due to the additional free reaction space.

Another result of the mode of operation according to the invention is that an overall plant for preparing cyclohexyl (meth)acrylate can be operated using a smaller amount of energy because of the reduced recycle streams. Furthermore, the lower thermal stressing of the target ester leads to a reduction in the formation of undesirable secondary components by decomposition of the target ester.

Under conventional distillation conditions, decomposition reactions, discoloration and unsatisfactory removal of the secondary components occur, such that the required purity of the cyclohexyl (meth)acrylate may not be satisfactory for a further use. The purification of the cyclohexyl (meth)acrylate is therefore an ongoing problem. This applies particularly to secondary components having a high boiling point, since these are separated off under conditions under which decomposition and product damage often associated therewith cannot be suppressed. Accordingly, it is extremely difficult to obtain the desired product in particular in a very high purity without great decreases in yield.

In the process described here, the temperature at the bottom of the column and thus the formation of secondary components can be reduced further according to the invention by the use of low-pressure-drop column internals.

Experiments on the thermal stability have shown that the formation of secondary components which are responsible for the deterioration in the product quality of the target ester increases with increasing temperature (see example 2 below).

A relatively low temperature at the bottom results, according to the invention, in the thermal stressing of the target ester being reduced and the product quality thus being increased and the formation of fouling in the vaporizers being decreased. The reduced fouling in the vaporizers leads to an increase in the time of operation and thereby to an increase in capacity.

As a result of the smaller recycle streams, the capacity of an overall plant for preparing cyclohexyl (meth)acrylate can, for example, be increased by about 15%.

Possible rectification columns are, for example, a column comprising random packing elements, a column comprising ordered packing or a tray column. The column is, as is generally every distillation column, equipped with a bottom vaporizer and a condenser at the top of the column.

The process is, according to the invention, carried out in a rectification column having a stripping section and an enrichment section and a bottom vaporizer. The bottom vaporizer is, in particular, a thin film evaporator which is preferably operated in the countercurrent mode, very particularly preferably a falling film evaporator which is preferably operated in the concurrent mode.

In the process of the present invention, the residence time in the bottom vaporizer and the associated piping system is particularly advantageously limited to from 1 to 60 minutes, preferably to from 10 to 30 minutes. In this way, problem-free operation of the plant, in particular only slight or no fouling, is ensured despite the polymerization capability of the mixture.

A preferred feature of the distillation process of the invention is that the vaporization is preferably carried out at a specific heat input in the range from 1 kW/m² to 100 kW/m², particularly preferably in the range from 2 kW/m² to 50 kW/m² and very particularly preferably from 5 kW/m² to 30 kW/m², with these figures being based on the heat introduced and the heated vaporizer surface of the vaporizer. Exceeding the upper value of 100 kW/m² leads, as has been recognized according to the invention, to increased fouling on the vaporizer surface because of the high temperature difference between product and heating medium which is required. Going below the lower value of 1 kW/m² leads to an uneconomical process since, at a given quantity of heat, the heated vaporizer surface area of the vaporizer would become too large or, at a given heated vaporizer surface area of the vaporizer, the quantities of heat to be transferred become too small.

In a preferred process variant, the liquid reflux ratio (i.e. ratio of amount of reflux and the amount of distillate taken off) of the column is regulated at a ratio of from 1:0.2 to 1:10 (i.e. from 5 to 0.1), more preferably at a ratio of from 1:0.2 to 1:1 (i.e. from 5 to 1). This is preferably effected by the liquid being collected downstream of the condenser and introduced via a regulating or setting device in the abovementioned ratio into the column and the distillate container, respectively. This ensures a lower energy consumption.

The process of the invention is preferably carried out at a pressure at the top of the column of from 10 mbar to 5 bar, preferably from 10 to 200 mbar, more preferably from 10 to 50 mbar.

The upper region of the column is preferably provided with temperature regulation having a measurement point below the uppermost theoretical plate, preferably at the third theoretical plate counted from the top, which utilizes the distillate flow, the reflux ratio or preferably the amount of reflux as manipulated variable. This ensures stable operation of the column, resulting in a further improvement in the achievable product purity.

In a further process variant, the lower region of the column is provided, additionally or as an alternative, with temperature regulation having a measurement point above the bottommost theoretical plate, preferably at the second theoretical plate counted from the bottom, which utilizes the amount taken off at the bottom as manipulated variable. A further improvement in the stable operation of the column is achieved by means of this additional measure.

The column has a number of theoretical plates of from 4 to 50, preferably from 5 to 20. The side feed stream for the mixture comprising cyclohexyl (meth)acrylate (the crude cyclohexyl (meth)acrylate) is introduced in the region commencing at least one theoretical plate above the bottommost theoretical plate and ending at least one theoretical plate below the uppermost theoretical plate.

At a number of theoretical plates of from 40 to 50, this feed stream is introduced preferably from 5 to 25 theoretical plates above the bottommost theoretical plate and from 5 to 25 theoretical plates below the uppermost theoretical plate.

At a number of theoretical plates of from 10 to 20, this feed stream is introduced preferably from 3 to 10 theoretical plates above the bottommost theoretical plate and from 3 to 10 theoretical plates below the uppermost theoretical plate.

For example, in the case of a column having from 45 to 50 theoretical plates, the feed for the crude cyclohexyl (meth)acrylate is preferably arranged at the theoretical plate between the 4th to 41st theoretical plate, and, in the case of a column having 20 theoretical plates, it is preferably arranged at the theoretical plate between the 2nd to 18th theoretical plate.

The mixture fed in at the side of the rectification column preferably has the following composition:

from 50 to 98% by weight, in particular from 75 to 98% by weight, e.g. from 76 to 98% by weight, of cyclohexyl (meth)acrylate,
from 0.5 to 20% by weight, in particular from 0.5 to 10% by weight, of cyclohexanol,
from 0.5 to 20% by weight, in particular from 0.5 to 8% by weight, of entrainer,
from 0.25 to 5% by weight, in particular from 0.25 to 3% by weight, of relatively high boilers [relative to cyclohexyl (meth)acrylate], from 0.25 to 5% by weight, in particular from 0.25 to 3% by weight, of further relatively low boilers [relative to cyclohexyl (meth)acrylate].

With respect to the separation-active internals, there are fundamentally no restrictions; random packing elements, ordered packing or trays are preferably provided.

In a preferred configuration, the internals are to be selected such that they bring about a specific pressure drop of from 0.05 to 10 mbar/theoretical plate, preferably from 0.1 to 5 mbar/theoretical plate.

The specific pressure drop is understood to mean the ratio of pressure drop of the internals and the number of theoretical plates.

In a preferred process variant, high-performance random packing elements are used as separation-active internals in the column.

High-performance random packing elements for processes of the chemical and related industry have a hydrodynamically advantageous basic shape having a high separation-active surface area per unit volume.

Compared to conventional random packing elements, a high-performance random packing element has both better hydrodynamic properties and a higher effectiveness in respect of mass transfer. It offers an extremely low pressure drop at the best mass transfer.

Examples of such high-performance random packing elements may be found in Chemie lngenieur Technik 2010, 82 No. 10, pages 1693 to 1703, (R. Billet et al.; DOI: 10.1002/cite.201000050).

The distillation unit in which the pure cyclohexyl (meth)acrylate is finally separated off via the top or via a side offtake is preferably a falling film evaporator, thin film evaporator or a distillation column.

The pure cyclohexyl acrylate obtained in the process of the invention, isolated from a mixture comprising cyclohexyl acrylate, cyclohexanol and entrainer, has, in particular, a purity of >98% by weight, very particularly preferably ≥98.5% by weight, more particularly 99% by weight.

The pure cyclohexyl methacrylate obtained in the process of the invention, isolated from a mixture comprising cyclohexyl methacrylate, cyclohexanol and entrainer, has, in particular, a purity of >98% by weight, very particularly preferably 98.5% by weight, more particularly 99% by weight.

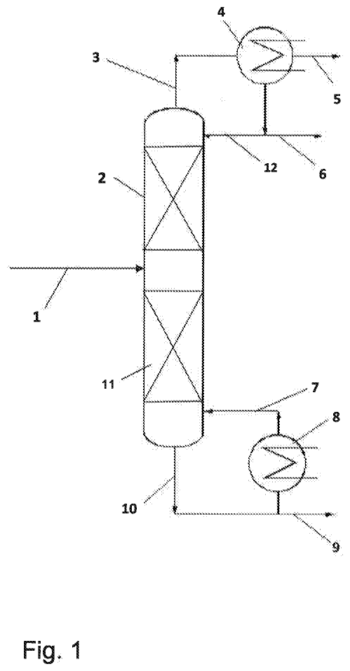

The invention will be illustrated in more detail below with the aid of a drawing (FIG. 1) and examples.

FIG. 1 shows the schematic depiction of a preferred rectification column for carrying out the process of the invention. The feed stream is conveyed via the feed conduit 1 into the column 2. The column is filled with random packing elements or ordered packing (11). The vapor stream 3 obtained at the top of the column is partially condensed in the condenser 4, which is optionally supplemented by an after-condenser, and divided into the reflux stream 12 and the distillate stream 6. The uncondensed fraction from the condenser 4 contains the low-boiling impurities and is taken off in vapor form as stream 5. At the lower end of the column, the liquid 10 is partially vaporized in a bottom vaporizer 8 and recirculated via the pipe 7 into the column. A main stream 9 which comprises the high-boiling impurities is taken off. The vaporizer 8 can be configured as a natural convection vaporizer or as a force circulation vaporizer; in the latter case, a circulation pump for the liquid stream 10 is additionally required. It is particularly advantageous, in terms of avoiding undesirable polymerization reactions, to use a falling film evaporator or thin film evaporator instead of the force circulation vaporizer since very short residence times are possible using evaporators of this type.

To reduce the residence time of the liquid in the vaporizer, it is advantageous to configure the bottom space including the bottom conduit in such a way that a very small liquid volume is present.

All pressures indicated are absolute pressures.

A ‘relatively low boiler’ [relative to cyclohexyl (meth)acrylate] is a material whose boiling point is lower than the boiling point of cyclohexyl acrylate or cyclohexyl methacrylate at the same pressure.

A ‘relatively high boiler’ [relative to cyclohexyl (meth)acrylate] is a material whose boiling point is higher than the boiling point of cyclohexyl (meth)acrylate or cyclohexyl methacrylate at the same pressure.

### EXAMPLES

### 1) Comparative Example

A crude cyclohexyl methacrylate was introduced at 883 kg/h and with a temperature of 97° C. into the column at the upper end of the random packing element bed made up of 25 mm Pall rings.

The composition of the crude cyclohexyl methacrylate was determined by means of analyses:

| | |
|---|---|
| cyclohexyl methacrylate | 95.2% by weight |
| cyclohexanol | 2.8% by weight |
| cyclohexane | 1.0% by weight |

further relatively low boilers, relatively high boilers: 1.0% by weight

The column was operated at a pressure at the top of 16 mbar and a pressure at the bottom of 37 mbar. At the top of the column, the distillate was condensed and collected in a container. This was continuously drained by means of level regulation, so that a continual reflux stream of distillate of 134 kg/h flows into the first esterification stage.

The composition of the distillate was determined by means of analyses and was:

| | |
|---|---|
| cyclohexyl methacrylate | 82% by weight |
| cyclohexanol | 11% by weight |
| cyclohexane | 5% by weight |
| further relatively low boilers | 2% by weight |

The bottoms from the column run through a falling film evaporator which is operated in countercurrent. The falling film evaporator was heated with 93 kW. The bottom product flowing out from the falling film evaporator at 746 kg/h had a temperature of 107° C.

The composition was determined by means of analyses:

| | |
|---|---|
| cyclohexyl methacrylate | 98% by weight |
| cyclohexanol | 1.3% by weight |
| relatively high boilers | 0.7% by weight |

The commercial specifications for cyclohexyl methacrylate were adhered to with 98% by weight of cyclohexyl methacrylate. The distillation yield of cyclohexyl methacrylate from this column was more than 86%.

### 2) Experiments on the Thermal Stability of a Feed Product (Side Feed of the Rectification Column)

Experiments on the thermal stability of a feed product, here a mixture of

94.2% by weight of cyclohexyl methacrylate,
2.5% by weight of cyclohexanol, 1.0% by weight of cyclohexene,
1.4% by weight of relatively high boilers and 0.9% by weight of further relatively low boilers,
at atmospheric pressure, three different temperatures (120, 130, 140° C.) and a residence time of one hour have shown that the formation of dimers and oligomers which are responsible for impairing the product quality of the target ester increases with increasing temperature.

In the following, the increase in the cyclohexyl methacrylate dimer of the formula

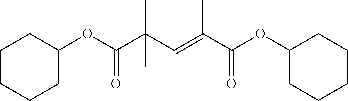 [SMILES: C/C(=C\C(C)(C)C(=O)OC1CCCCC1)C(=O)OC1CCCCC1]

is shown versus a temperature increase (gas-chromatographic analysis);

| | |
|---|---|
| Starting material: | 0.52 percent by area |
| 120° C. | 0.56 percent by area |
| 130° C. | 0.64 percent by area |
| 140° C. | 0.69 percent by area |

### 3) Example According to the Invention

The mode of operation is shown with the aid of data from a thermodynamic simulation of an overall plant for preparing cyclohexyl methacrylate.

The thermodynamic simulation of the process was carried out using the software Aspen Plus® (Aspen for short). Aspen is comprehensive simulation software which is used for modeling, simulation and optimization of chemical processes and plants in industry. Aspen has comprehensive modeling data banks for modeling the basic operations and also materials data banks for the materials properties of many different substances. The properties of mixtures are calculated by Aspen from the materials data of the pure substances by means of various thermodynamic models.

The thermodynamic simulation of the overall plant led to the following results:

A crude cyclohexyl methacrylate having a temperature of 97° C. is introduced at 883 kg/h into a rectification column having 12 theoretical plates at the 5th theoretical plate.

The composition of the inflowing crude cyclohexyl methacrylate is:

| | |
|---|---|
| cyclohexyl methacrylate | 94.7% by weight |
| cyclohexanol | 2.7% by weight |
| cyclohexane | 1% by weight |
| further relatively low boilers | 0.7% by weight |
| relatively high boilers | 0.9% by weight |

The column is operated at a pressure at the top of 16 mbar and a pressure at the bottom of 37 mbar. The specific pressure drop in the column is 1.75 mbar/theoretical plate. The temperature at the top is 61° C. At the top of the column, the distillate is condensed. The reflux stream of distillate in the first esterification stage amounts to 40 kg/h. The column is operated at a reflux ratio of 3.1.

The composition of the distillate is:

| | |
|---|---|
| cyclohexyl methacrylate | 10% by weight |
| cyclohexanol | 59% by weight |
| cyclohexane | 15% by weight |
| further relatively low boilers | 16% by weight |

The vaporizer is operated at 31 kW. The bottom product flowing out from the vaporizer at 840 kg/h has a temperature of 107° C.

The composition of the bottom output is:

| | |
|---|---|
| cyclohexyl methacrylate | 99% by weight |
| cyclohexanol | 0.1% by weight |
| relatively high boilers | 0.9% by weight |

The distillation of crude cyclohexyl (meth)acrylate can be carried out by means of the process of the invention at the same daily capacity of, for example, 14.0 metric tons while adhering to the required specifications with an energy cost saving of 69% compared to a conventional distillation process.

The reduction in the recirculation to the esterification from 134 kg/h to 40 kg/h enables the daily capacity of an overall plant for preparing cyclohexyl (meth)acrylate to be increased, for example, by about 100 kg/h, corresponding to a capacity increase of about 14%.

As a result of the lowering of the temperature at the bottom of the vaporizer from 113° C. to 107° C., the formation of unknown secondary components which are responsible for the fouling in the vaporizer is considerably reduced.

Since the time of operation of an overall plant for preparing cyclohexyl (meth)acrylate is limited by the time of operation of the vaporizer as a result of fouling, the time of operation of an overall plant can be increased by the reduction in secondary component formation due to the reduced temperature at the bottom in the low boiler distillation. This leads to a higher annual capacity of the overall plant.

1. A process for isolating pure cyclohexyl (meth)acrylate from a mixture comprising cyclohexyl (meth)acrylate, cyclohexanol and entrainer by distillation, wherein the mixture is fed as side feed stream into a rectification column having a bottom vaporizer, separation-active internals and a stripping section and an enrichment section, where the column has from 4 to 50 theoretical plates, the side feed stream is introduced in the region commencing at least one theoretical plate above the bottommost theoretical plate and ending at least one theoretical plate below the uppermost theoretical plate, the pressure at the top of the column is in the range from 10 mbar to 5 bar and the reflux ratio is from 1:0.2 to 1:10, and the bottom output is fed to a further distillation unit in which the cyclohexyl (meth)acrylate is separated off at the top or at a side offtake.

2. The process according to claim 1, wherein the entrainer is cyclohexane.

3. The process according to claim 1, wherein the separation-active internals are random packing elements, ordered packing or trays.

4. The process according to claim 1, wherein the separation-active internals bring about a specific pressure drop in the range from 0.05 to 10 mbar per theoretical plate.

5. The process according to claim 1, wherein the random packing elements are high-performance random packing elements.

6. The process according to claim 1, wherein the residence time in the bottom vaporizer and the associated piping system is in the range from 1 to 60 minutes.

7. The process according to claim 1, wherein the residence time in the bottom vaporizer and the associated piping system is in the range from 10 to 30 minutes.

8. The process according to claim 1, wherein the bottom vaporizer is a thin film evaporator or falling film evaporator.

9. The process according to claim 1, wherein the vaporization in the bottom vaporizer is effected at a specific heat input in the range from 1 to 100 kW/m².

10. The process according to claim 1, wherein the vaporization in the bottom vaporizer is effected at a specific heat input in the range from 2 to 50 kW/m².

11. The process according to claim 1, wherein the vaporization in the bottom vaporizer is effected at a specific heat input in the range from 5 to 30 kW/m².

12. The process according to claim 1, wherein the column has from 5 to 20 theoretical plates and the side feed stream is introduced in the region commencing at least one theoretical plate above the bottommost theoretical plate and ending at least one theoretical plate below the uppermost theoretical plate.

13. The process according to claim 1, wherein the pressure at the top of the column is in the range from 10 mbar to 200 mbar.

14. The process according to claim 1, wherein the reflux ratio is from 1:0.2 to 1:1.

15. The process according to claim 1, wherein
the mixture fed in at the side of the rectification column has the following composition:
from 50 to 98% by weight of cyclohexyl (meth)acrylate,
from 0.5 to 20% by weight of cyclohexanol,
from 0.5 to 20% by weight of entrainer,
from 0.25 to 5% by weight of relatively high boilers [relative to cyclohexyl (meth)acrylate], from 0.25 to 5% by weight of further relatively low boilers [relative to cyclohexyl (meth)acrylate].

16. The process according to claim 1, wherein the process is for isolating pure cyclohexyl acrylate having a purity of >98% by weight from a mixture comprising cyclohexyl acrylate, cyclohexanol and entrainer.

17. The process according to claim 1, wherein the process is for isolating pure cyclohexyl methacrylate having a purity of >98% by weight from a mixture comprising cyclohexyl methacrylate, cyclohexanol and entrainer.
